(19) United States
(12) Patent Application Publication
(10) Pub. No.: US 2016/0066970 A1
(43) Pub. Date: Mar. 10, 2016

(54) BONE FIXATION APPARATUS

(71) Applicant: THE CLEVELAND CLINIC FOUNDATION, Cleveland, OH (US)

(72) Inventor: Thomas J. Graham, Novelty, OH (US)

(21) Appl. No.: 14/944,378

(22) Filed: Nov. 18, 2015

Related U.S. Application Data
(63) Continuation of application No. 13/933,333, filed on Jul. 2, 2013.
Application No. 13/933,333 is a continuation of application No. 13/367,328, filed on Feb. 6, 2012.
(60) Provisional application No. 61/667,009, filed on Jul. 2, 2012.
(60) Provisional application No. 61/583,527, filed on Jan. 5, 2012.

(51) Int. Cl. A61B 17/80

(52) U.S. Cl. CPC A61B 17/8085; A61B 2017/00946

(57) ABSTRACT

A bone fixation apparatus comprises a plurality of boundary portions. Each boundary portion defines and surrounds an opening for a fastener. The bone fixation apparatus also comprises a plurality of interconnecting portions. Each interconnecting portion has two opposed ends and a length extending between the opposed ends. Each interconnecting portion is joined at one of its opposed ends to a first boundary portion and at the other of its opposed ends to a second boundary portion so as to extend radially from the first and second boundary portions. Each boundary portion is joined only to at least one interconnecting portion and is free of any structure extending radially from the boundary portion other than the at least one interconnecting portion. Three of the plurality of boundary portions and two of the interconnecting portions are arranged in a zigzag configuration with one of the three boundary portions being located in a central position. The two interconnecting portions extend radially from the boundary portion in the central position to two different ones of the three boundary portions. The boundary portion in the central position is disposed in a first plane. The two different ones of the three boundary portions are disposed in a second plane.

### CROSS-REFERENCE TO RELATED APPLICATION

This application claims the benefit of U.S. Provisional Patent Application No. 61/667,009, filed Jul. 2, 2012 and entitled BONE FIXATION APPARATUS, which is incorporated herein by reference in its entirety.

### FIELD OF THE INVENTION

The present invention relates to a bone fixation apparatus comprising boundary portions that define fastener openings and interconnecting portions that join the boundary portions and, more particularly, to a bone fixation apparatus in which interconnecting portions join boundary portions disposed in different planes.

### BACKGROUND OF THE INVENTION

One method for treating a fractured bone is to apply an internal device to fix the broken pieces of bone in position. Such an internal fixation device may be, for example, a bone plate, bone screw, or nail. When fixing the broken pieces of a bone in position, it is important to align the pieces properly. This may be difficult to accomplish if one or more of the bone pieces is small or displaced from its proper position or if the fracture extends in a spiral along the bone.

### SUMMARY OF THE INVENTION

The present invention is directed to a bone fixation apparatus comprising boundary portions that define fastener openings and interconnecting portions that join the boundary portions and, more particularly, to a bone fixation apparatus in which interconnecting portions join boundary portions disposed in different planes.

In accordance with an embodiment of the present invention, a bone fixation apparatus comprises a plurality of boundary portions. Each boundary portion defines and surrounds an opening for a fastener. The bone fixation apparatus also comprises a plurality of interconnecting portions. Each interconnecting portion has two opposed ends and a length extending between the opposed ends. Each interconnecting portion is joined at one of its opposed ends to a first boundary portion and at the other of its opposed ends to a second boundary portion so as to extend radially from the first and second boundary portions. Each boundary portion is joined only to at least one interconnecting portion and is free of any structure extending radially from the boundary portion other than the at least one interconnecting portion. Three of the plurality of boundary portions and two of the interconnecting portions are arranged in a zigzag configuration with one of the three boundary portions being located in a central position. The two interconnecting portions extend radially from the boundary portion in the central position to two different ones of the three boundary portions. The boundary portion in the central position is disposed in a first plane. The two different ones of the three boundary portions are disposed in a second plane.

In accordance with another embodiment of the present invention, a bone fixation apparatus comprises a plurality of boundary portions. Each boundary portion defines and surrounds an opening for a fastener. The bone fixation apparatus also comprises a plurality of interconnecting portions. Each interconnecting portion has two opposed ends and a length extending between the opposed ends. Each interconnecting portion is joined at one of its opposed ends to a first boundary portion and at the other of its opposed ends to a second boundary portion so as to extend radially from the first and second boundary portions. Each boundary portion is joined only to at least one interconnecting portion and is free of any structure extending radially from the boundary portion other than the at least one interconnecting portion. Five of the plurality of boundary portions and four of the interconnecting portions are arranged in an X configuration. One of the five boundary portions is located in a central position. The four interconnecting portions extend radially from the boundary portion in the central position to four different ones of the five boundary portions. The boundary portion in the central position is disposed in a first plane. The four different ones of the five boundary portions are disposed in second planes transverse to the first plane.

In accordance with a further embodiment of the present invention, a bone fixation apparatus comprises a plurality of boundary portions. (a) a plurality of boundary portions. Each boundary portion defines and surrounds an opening for a fastener. The bone fixation apparatus also comprises a plurality of interconnecting portions. Each interconnecting portion has two opposed ends and a length extending between the opposed ends. Each interconnecting portion is joined at one of its opposed ends to a first boundary portion and at the other of its opposed ends to a second boundary portion so as to extend radially from the first and second boundary portions. The bone fixation apparatus further comprises a plurality of tab portions. Each tab portion extends radially from a single boundary portion. Each boundary portion is joined only to at least one of the tab portions and the interconnecting portions. Each boundary portion also is free of any structure extending radially from said each boundary portion other than said at least one of the tab portions and the interconnecting portions. Three of the plurality of boundary portions and two of the interconnecting portions are arranged in a zigzag configuration with one of the three boundary portions being located in a central position and the two interconnecting portions extending radially from the boundary portion in the central position to two different ones of the three boundary portions. The boundary portion in the central position is disposed in a first plane and the two different ones of the three boundary portions are disposed in a second plane. One of the tab portions extends radially from one of the two different ones of the three boundary portions and is at least partially disposed in a third plane spaced from and generally parallel to the first plane.

### BRIEF DESCRIPTION OF THE DRAWINGS

The foregoing and other features and advantages of the present invention will become apparent to one skilled in the art upon consideration of the following description of the invention and the accompanying drawings, in which:

FIG. 1 is a top view of a bone fixation apparatus, in accordance with an embodiment of the present invention, applied to a fractured bone;

FIG. 2 is a side view of the bone fixation apparatus of FIG. 1;

FIG. 3 is a top view of a bone fixation apparatus, in accordance with a second embodiment of the present invention, applied to a fractured bone;

FIG. 4 is a side view of the bone fixation apparatus of FIG. 3;

FIG. 5 is a side view of a bone fixation apparatus, in accordance with a third embodiment of the present invention, applied to a fractured bone;

FIG. 6 is a sectional view of a portion of the bone fixation apparatus of FIG. 5, taken along line 6-6 of FIG. 5;

FIG. 7 is a top view of a bone fixation apparatus, in accordance with a fourth embodiment of the present invention; and

FIG. 8 is a top view of a modified version of the bone fixation apparatus of FIG. 7.

### DETAILED DESCRIPTION

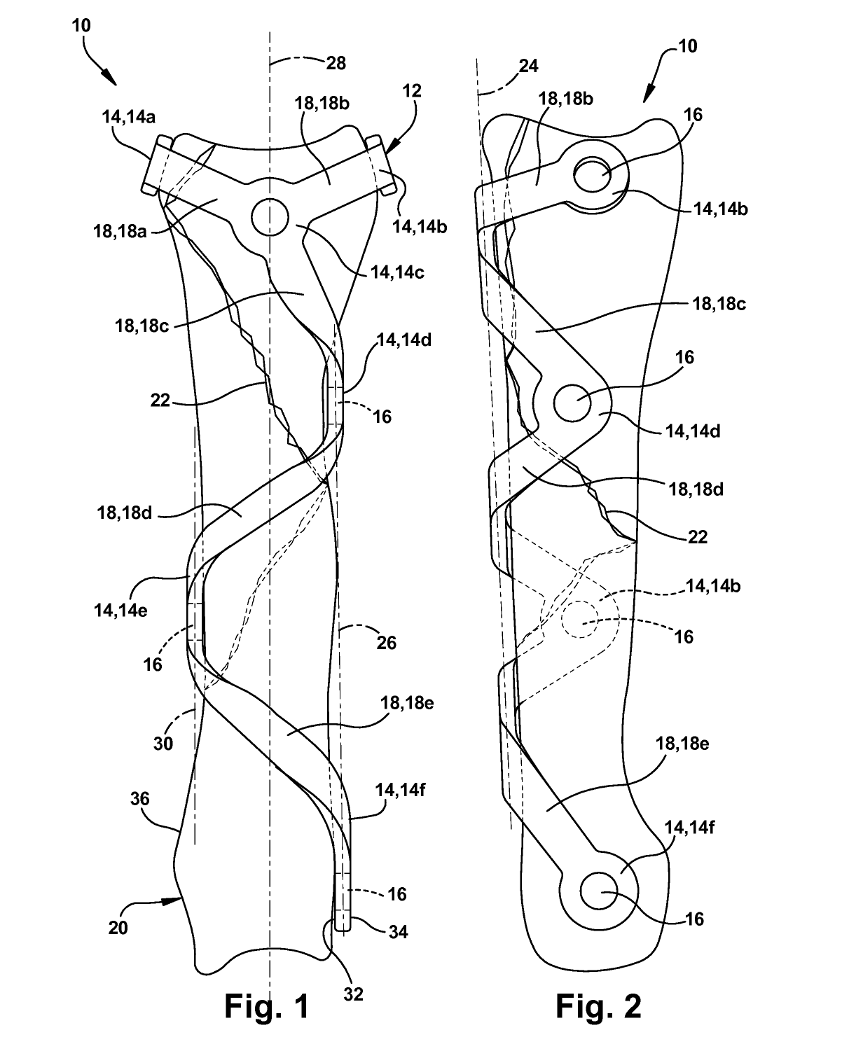

FIGS. 1 and 2 illustrate a bone fixation apparatus 10 in accordance with an example of the present invention. The bone fixation apparatus 10 comprises a bone plate 12 that includes a plurality of boundary portions 14. Each boundary portion 14 defines and surrounds an opening 16 that is configured and dimensioned to receive a fastener, such as a bone screw (not shown). The bone plate 12 of the bone fixation apparatus 10 also includes plurality of interconnecting portions 18 that join two boundary portions 14 to one another. As shown in FIGS. 1 and 2, the bone fixation apparatus 10 may be applied to a bone 20 to reduce and stabilize a fracture 22 in the bone. In the bone 20, the fracture 22 is a spiral fracture that has caused the cortical portion of the bone to split along a spiral line.

In the embodiment of FIGS. 1 and 2, the plurality of boundary portions 14 in the bone plate 12 comprises six boundary portions 14a, 14b, 14c, 14d, 14e, and 14f. The plurality of interconnecting portions 18 in the bone plate 12 comprises five interconnecting portions 18a, 18b, 18c, 18d, and 18e. Each of interconnecting portions 18a-18e has two opposed ends and a length extending between the opposed ends. Each of the interconnecting portions 18a-18e is joined at one of its opposed ends to a first boundary portion 14 and at the other of its opposed ends to second boundary portion. For example, the interconnecting portion 18a is joined at one end to the boundary portion 14a and at its other end to the boundary portion 14c. The six boundary portions 14 and the five interconnecting portions 18 are all formed in one piece from a homogeneous material. The material is a relatively rigid bio-compatible material, such as medical grade stainless steel or titanium.

The boundary portions 14 and interconnecting portions 18 are joined together in an array that provides the bone plate 12 with a generally zigzag configuration. At one end of the bone plate 12, the boundary portions 14a and 14b are disposed adjacent opposite sides of the bone 20. As a consequence, the boundary portions 14a and 14b are spaced apart by the thickness or width of the bone 20 between the boundary portions. The boundary portions 14a and 14b are also presented generally toward one another. The boundary portion 14c is positioned laterally between the boundary portions 14a and 14b and is spaced longitudinally from the boundary portions 14a and 14b along the length of the bone plate 12 and the length of the bone 20. The boundary portion 14c also extends laterally in a plane 24 transverse to the planes in which the boundary portions 14a and 14b extend.

As previously described, the interconnecting portion 18a extends between and joins together the boundary portion 14a and the boundary portion 14c. The interconnecting portion 18b extends between and joins together the boundary portion 14b and the boundary portion 14c. Because the boundary portion 14c lies in a plane 24 that extends transverse to the planes in which the boundary portions 14a and 14b lie, the interconnecting portions 18a and 18b curve or bend around the bone 20. In addition, because the boundary portion 14c is spaced apart from the boundary portions 14a and 14b along the length of the bone plate 12 and the length of the bone 20, the interconnecting portions 18a and 18b extend across an exterior surface 36 of the bone at angles to the longitudinal central axis 28 of the bone plate and the bone. As viewed in FIG. 1, the interconnecting portion 18a zigs to the right, and the interconnecting portion 18b zags to the left.

The boundary portion 14d is spaced from the boundary portion 14c along the length of the bone plate 12 and the length of the bone 20 and is disposed in a plane 26 adjacent the right side of the bone plate and the bone, as viewed in FIG. 1. The interconnecting portion 18c extends between and joins together the boundary portion 14c and the boundary portion 14d. Because the boundary portion 14d lies in a plane 26 that extends transverse to the plane 24 in which the boundary portion 14c lies, the interconnecting portion 18c curves or bends around the bone 20. In addition, because the boundary portion 14d is spaced apart from the boundary portion 14c along the length of the bone plate 12 and the length of the bone 20, the interconnecting portion 18c extends across the exterior surface 36 of the bone at an angle to the longitudinal central axis 28 of the bone plate and the bone. As viewed in FIG. 1, the interconnecting portion 18c zigs to the right.

The boundary portion 14e is spaced from the boundary portion 14d along the length of the bone plate 12 and the length of the bone 20 and is disposed in a plane 30 adjacent the left side of the bone plate and the bone, as viewed in FIG. 1. The plane 30 is spaced laterally apart from the plane 26 by the width of the bone plate 12 and the thickness or width of the bone 20, but the planes 26 and 30 are substantially parallel to one another. The interconnecting portion 18d extends between and joins together the boundary portion 14d and the boundary portion 14e. Because the boundary portion 14e lies in a plane 30 that is spaced laterally from the plane 26 in which the boundary portion 14d lies, the interconnecting portion 18c curves or bends around the bone 20. In addition, because the boundary portion 14e is spaced apart from the boundary portion 14d along the length of the bone plate 12 and the length of the bone 20, the interconnecting portion 18d extends across the exterior surface 36 of the bone at an angle to the longitudinal central axis 28 of the bone plate and the bone. As viewed in FIG. 1, the interconnecting portion 18d zags to the left.

The boundary portion 14f is spaced from the boundary portion 14d and the boundary portion 14e along the length of the bone plate 12 and the length of the bone 20 and is disposed in the plane 26 adjacent the right side of the bone plate and the bone, as viewed in FIG. 1. The interconnecting portion 18e extends between and joins together the boundary portion 14e and the boundary portion 14f. Because the boundary portion 14e lies in a plane 30 that is spaced laterally from the plane 26 in which the boundary portion 14f lies, the interconnecting portion 18e curves or bends around the bone 20. In addition, because the boundary portion 14f is spaced apart from the boundary portion 14e along the length of the bone plate 12 and the bone 20, the interconnecting portion 18e extends across the exterior surface 36 of the bone at an angle to the longitudinal central axis 28 of the bone plate and the bone. As viewed in FIG. 1, the interconnecting portion 18e zigs to the right.

As viewed in FIG. 1, boundary portion 14e is located in a central position relative to the boundary portions 14d and 14f along the length of the bone plate 12 and the length of the bone 20. The interconnecting portions 18d and 18e extend radially from the boundary portion 14e. Each of the boundary portions 14a-14f is joined only to one or more of the interconnecting portions 18a-18e. Each of the boundary portions 14a-14f is free of any structure extending radially from the boundary portion other than one or more of the interconnecting portions 18a-18e.

Although the bone plate 12 has a zigzag configuration and at least partially wraps around the bone 20, the bone plate maintains an inward facing surface 32 configured to be presented toward the external surface 36 of the bone 20. Opposite the inward facing surface 32 is an outward facing surface 34, which is configured to be presented away from the external surface 36 of the bone 20. The openings 16 defined by the boundary portions 14 all extend from the outward facing surface 34 completely through the bone plate 12 to the inward facing surface 32. As mentioned above, the openings 16 are configured and dimensioned to receive fasteners (not shown), such as bone screws, to help secure the bone plate 12 to the bone 20.

In use, the bone plate 12 of the bone fixation apparatus 10 is first placed against an external surface of a fractured bone. With particular reference to FIGS. 1-2, the bone plate 12 is placed against the bone 20 such that the inward facing surface 32 of the bone plate contacts the external surface 36 of the bone. The position of the bone plate 12 is then adjusted to facilitate attachment of the bone plate to the bone 20 and to facilitate desired placement of the bone plate. After the desired position of the bone plate 12 is determined, the bone plate may be moved to allow the bone 20 to be drilled and/or otherwise prepared to receive the bone plate. After the bone 20 has been prepared, the bone plate 12 is moved to its desired position and fasteners (not shown), such as bone screws, are installed to attach the bone plate to the bone.

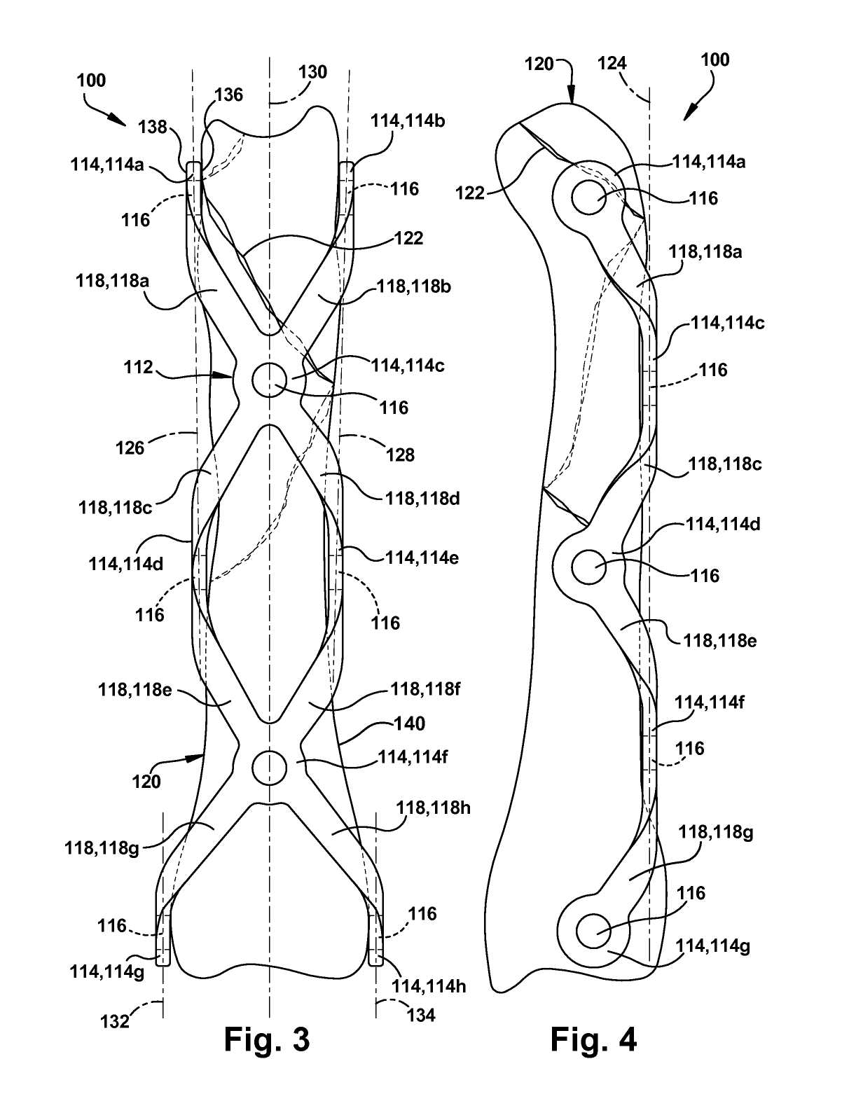

FIGS. 3 and 4 illustrate a bone fixation apparatus 100 in accordance with a second example of the present invention. The bone fixation apparatus 100 comprises a bone plate 112 that includes a plurality of boundary portions 114. Each boundary portion 114 defines and surrounds an opening 116 that is configured and dimensioned to receive a fastener, such as a bone screw (not shown). The bone plate 112 of the bone fixation apparatus 100 also includes plurality of interconnecting portions 118 that join two boundary portions 114 to one another. As shown in FIGS. 3 and 4, the bone fixation apparatus 100 may be applied to a bone 120 to reduce and stabilize a fracture 122 in the bone. In the bone 120, the fracture 122 is a spiral fracture that has caused the cortical portion of the bone to split along a spiral line.

In the embodiment of FIGS. 3 and 4, the plurality of boundary portions 114 in the bone plate 112 comprises eight boundary portions 114a, 114b, 114c, 114d, 114e, 114f, 114g, and 114h. The plurality of interconnecting portions 118 in the bone plate 112 comprises eight interconnecting portions 118a, 118b, 118c, 118d, 118e, 118f, 118g, and 118h. Each of interconnecting portions 118a-118h has two opposed ends and a length extending between the opposed ends. Each of the interconnecting portions 118a-118h is joined at one of its opposed ends to a first boundary portion 114 and at the other of its opposed ends to second boundary portion. For example, the interconnecting portion 118a is joined at one end to the boundary portion 114a and at its other end to the boundary portion 114c. The eight boundary portions 114 and the eight interconnecting portions 118 are all formed in one piece from a homogeneous material. The material is a relatively rigid bio-compatible material, such as medical grade stainless steel or titanium.

The boundary portions 114 and interconnecting portions 118 are joined together in an array that provides the bone plate 112 with a longitudinal series of configurations shaped like X's or, viewed differently, with a pair of zigzag configurations. In particular, the boundary portions 114a and 114b are disposed adjacent opposed sides of the bone 120. As a consequence, the boundary portions 114a and 114b are spaced apart by the thickness or width of the bone 120 between the boundary portions. The boundary portions 114a and 114b are also presented generally toward one another. The boundary portion 114c is positioned laterally between the boundary portions 114a and 114b and is spaced longitudinally from the boundary portions 114a and 114b along the length of the bone plate 112 and the length of the bone 120. The boundary portion 114c also extends laterally in a plane 124 transverse to the planes 126 and 128 in which the boundary portions 114a and 114b, respectively, extend.

As previously described, the interconnecting portion 118a extends between and joins together the boundary portion 114a and the boundary portion 114c. The interconnecting portion 118b extends between and joins together the boundary portion 114b and the boundary portion 114c. Because the boundary portion 114c lies in a plane 124 that extends transverse to the planes 126 and 128 in which the boundary portions 114a and 114b lie, the interconnecting portions 118a and 118b curve or bend around the bone 120. In addition, because the boundary portion 114c is spaced apart from the boundary portions 114a and 114b along the length of the bone plate 112 and the length of the bone 120, the interconnecting portions 118a and 118b extend across an exterior surface 140 of the bone at angles to the longitudinal central axis 130 of the bone plate and the bone. As viewed in FIG. 3, the interconnecting portion 118a zigs to the right, and the interconnecting portion 118b zags to the left.

The boundary portion 114d is spaced from the boundary portion 114c along the length of the bone plate 112 and the length of the bone 120 and is disposed in the plane 126 adjacent the left side of the bone plate and the bone, as viewed in FIG. 3. The interconnecting portion 118c extends between and joins together the boundary portion 114c and the boundary portion 114d. Because the boundary portion 114d lies in a plane 126 that extends transverse to the plane 124 in which the boundary portion 114c lies, the interconnecting portion 118c curves or bends around the bone 120. In addition, because the boundary portion 114d is spaced apart from the boundary portion 114c along the length of the bone plate 112 and the length of the bone 120, the interconnecting portion 118c extends across the exterior surface 140 of the bone at an angle to the longitudinal central axis 130 of the bone plate and the bone. As viewed in FIG. 3, the interconnecting portion 118c zags to the left.

The boundary portion 114e is spaced longitudinally from the boundary portion 114c along the length of the bone plate 112 and the length of the bone 120 and is disposed in the plane 128 adjacent the right side of the bone plate and the bone, as viewed in FIG. 3. The boundary portion 114e is spaced laterally from the boundary portion 114d across the width of the bone plate 112 and the thickness or width of the bone 120 but is aligned with the boundary portion 114d longitudinally along the length of the bone plate 112 and the length of the bone 120, as can be seen in FIG. 3. The plane 128 in which the boundary portion 114e is disposed is spaced laterally apart from the plane 126 in which the boundary portion 114d is disposed by the width of the bone plate 112 and thickness or width of the bone 120, but the planes 126 and 128 are substantially parallel to one another. The plane 128 in which the boundary portion 114e is disposed is transverse, however, to the plane 124 in which the boundary portion 114c is disposed.

The interconnecting portion 118d extends between and joins together the boundary portion 114c and the boundary portion 114e. Because the boundary portion 114e lies in a plane 128 that is transverse to the plane 124 in which the boundary portion 114c lies, the interconnecting portion 118d curves or bends around the bone 120. In addition, because the boundary portion 114e is spaced apart from the boundary portion 114c along the length of the bone plate 112 and the length of the bone 120, the interconnecting portion 118d extends across the exterior surface 140 of the bone at an angle to the longitudinal central axis 130 of the bone plate and the bone. As viewed in FIG. 3, the interconnecting portion 118d zigs to the right.

The boundary portion 114f is spaced from the boundary portions 114d and the boundary portion 114e along the length of the bone plate 112 and the length of the bone 120 and is disposed in the plane 124 in which the boundary portion 114c is disposed. The interconnecting portion 118e extends between and joins together the boundary portion 114d and the boundary portion 114f. Because the boundary portion 114d lies in a plane 126 that is transverse to the plane 124 in which the boundary portion 114f lies, the interconnecting portion 118e curves or bends around the bone 120. In addition, because the boundary portion 114f is spaced apart from the boundary portion 114d along the length of the bone plate 112 and the bone 120, the interconnecting portion 118e extends across the exterior surface 140 of the bone at an angle to the longitudinal central axis 130 of the bone plate and the bone. As viewed in FIG. 3, the interconnecting portion 118e zigs to the right.

The interconnecting portion 118f extends between and joins together the boundary portion 114e and the boundary portion 114f. Because the boundary portion 114e lies in a plane 128 that is transverse to the plane 124 in which the boundary portion 114f lies, the interconnecting portion 118f curves or bends around the bone 120. In addition, because the boundary portion 114f is spaced apart from the boundary portion 114e along the length of the bone plate 112 and the bone 120, the interconnecting portion 118f extends across the exterior surface 140 of the bone at an angle to the longitudinal central axis 130 of the bone plate and the bone. As viewed in FIG. 3, the interconnecting portion 118f zags to the left.

The boundary portion 114g is spaced longitudinally from the boundary portion 114f along the length of the bone plate 112 and the length of the bone 120 and is disposed in a plane 132 adjacent the left side of the bone plate and the bone, as viewed in FIG. 3. The plane 132 is generally parallel to, but spaced laterally from the plane 126. The interconnecting portion 118g extends between and joins together the boundary portion 114f and the boundary portion 114g. Because the boundary portion 114g lies in a plane 132 that extends transverse to the plane 124 in which the boundary portion 114f lies, the interconnecting portion 118g curves or bends around the bone 120. In addition, because the boundary portion 114g is spaced apart from the boundary portion 114f along the length of the bone plate 112 and the length of the bone 120, the interconnecting portion 118g extends across the exterior surface 140 of the bone at an angle to the longitudinal central axis 130 of the bone plate and the bone. As viewed in FIG. 3, the interconnecting portion 118g zags to the left.

The boundary portion 114h is spaced longitudinally from the boundary portion 114f along the length of the bone plate 112 and the length of the bone 120 and is disposed in a plane 134 adjacent the right side of the bone plate and the bone, as viewed in FIG. 3. The plane 134 is generally parallel to, but spaced laterally from the plane 128. The boundary portion 114h is spaced laterally from the boundary portion 114g across the width of the bone plate 112 and the thickness or width of the bone 120 but is aligned with the boundary portion 114g longitudinally along the length of the bone plate 112 and the length of the bone 120, as can be seen in FIG. 3. The plane 134 in which the boundary portion 114h is disposed is spaced laterally apart from the plane 132 in which the boundary portion 114g is disposed by the width of the bone plate 112 and the thickness or width of the bone 120, but the planes 132 and 134 are substantially parallel to one another. The plane 134 in which the boundary portion 114h is disposed is transverse, however, to the plane 124 in which the boundary portion 114f is disposed.

The interconnecting portion 118h extends between and joins together the boundary portion 114f and the boundary portion 114h. Because the boundary portion 114h lies in a plane 134 that is transverse to the plane 124 in which the boundary portion 114f lies, the interconnecting portion 118h curves or bends around the bone 120. In addition, because the boundary portion 114h is spaced apart from the boundary portion 114f along the length of the bone plate 112 and the length of the bone 120, the interconnecting portion 118h extends across the exterior surface 140 of the bone at an angle to the longitudinal central axis 130 of the bone plate and the bone. As viewed in FIG. 3, the interconnecting portion 118h zigs to the right.

As viewed in FIG. 3, boundary portion 114c is located in a central position relative to the boundary portions 114a, 114b, 114d, and 114e along the length of the bone plate 112 and the length of the bone 120. The interconnecting portions 118a, 118b, 118c, and 118d extend radially from the boundary portion 114c. The boundary portions 114a, 114b, 114c, 114d, and 114e and the interconnecting portions 118a, 118b, 118c, and 118d are thus arranged in a configuration shaped like an “X”. In addition, the boundary portions 114a, 114c, and 114d and the interconnecting portions 118a and 118c are arranged in a zigzag configuration. Further, the boundary portions 114b, 114c, and 114e and the interconnecting portions 118b and 118d are arranged in a zigzag configuration that is opposed to the zigzag configuration of the boundary portions 114a, 114c, and 114d and the interconnecting portions 118a and 118c. Each of the boundary portions 114a-114e is joined only to one or more of the interconnecting portions 118a-118f. Each of the boundary portions 114a-114e is free of any structure extending radially from the boundary portion other than one or more of the interconnecting portions 118a-118f.

Similarly, as viewed in FIG. 3, boundary portion 114f is located in a central position relative to the boundary portions 114d, 114e, 114g, and 114h along the length of the bone plate 112 and the length of the bone 120. The interconnecting portions 118e, 118f, 118g, and 118h extend radially from the boundary portion 114f. The boundary portions 114d, 114e, 114f, 114g, and 114h and the interconnecting portions 118e, 118f, 118g, and 118h are thus arranged in a configuration shaped like an “X”. In addition, the boundary portions 114d, 114f, and 114g and the interconnecting portions 118e and 118g are arranged in a zigzag configuration. Further, the boundary portions 114e, 114f, and 114h and the interconnecting portions 118f and 118h are arranged in a zigzag configuration that is opposed to the zigzag configuration of the boundary portions 114d, 114f, and 114g and the interconnecting portions 118e and 118g. Each of the boundary portions 114d-114h is joined only to one or more of interconnecting portions 118c-118h. Each of the boundary portions 114d-114h is free of any structure extending radially from the boundary portion other than one or more of interconnecting portions 118c-118h.

Although the bone plate 112 at least partially wraps around the bone 120, the bone plate maintains an inward facing surface 136 configured to be presented toward the external surface 140 of the bone 120. Opposite the inward facing surface 136 is an outward facing surface 138, which is configured to be presented away from the external surface 140 of the bone 120. The openings 116 defined by the boundary portions 114 all extend from the outward facing surface 138 completely through the bone plate 112 to the inward facing surface 136. As mentioned above, the openings 116 are configured and dimensioned to receive fasteners (not shown), such as bone screws, to help secure the bone plate 112 to the bone 120.

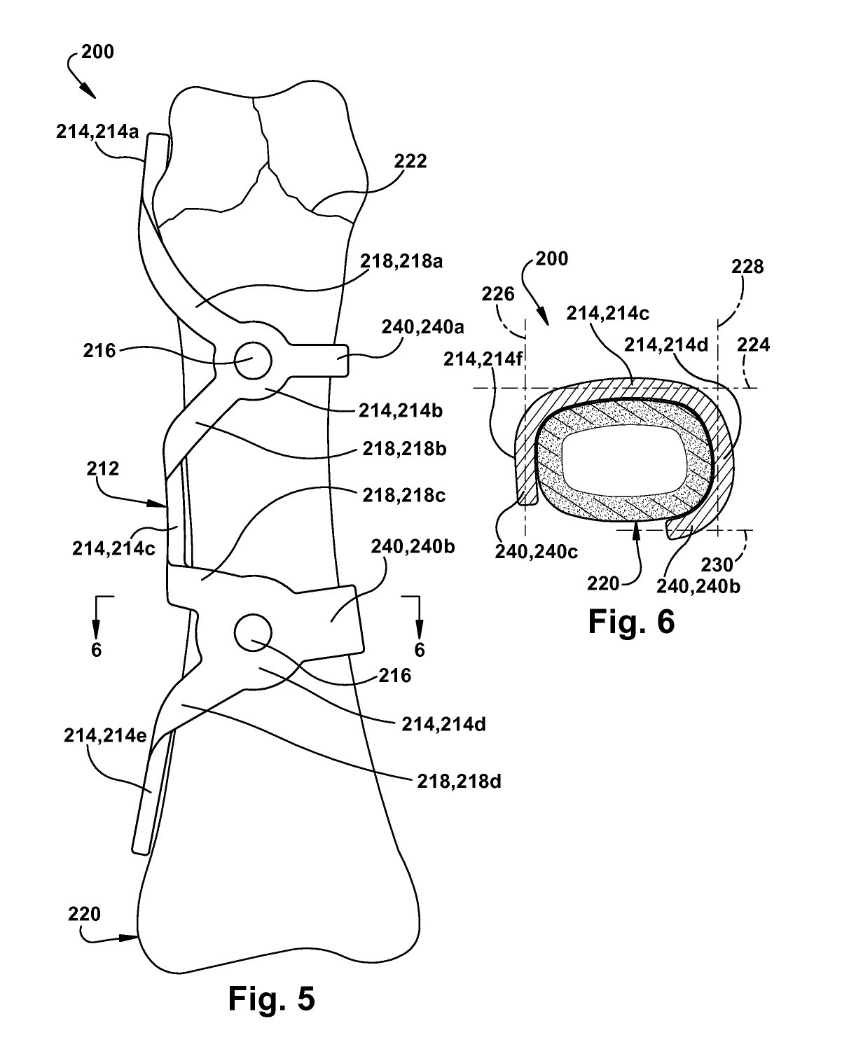

FIGS. 5 and 6 illustrate a bone fixation apparatus 200 in accordance with a third example of the present invention. The bone fixation apparatus 200 comprises a bone plate 212 that includes a plurality of boundary portions 214. Each boundary portion 214 defines and surrounds an opening 216 that is configured and dimensioned to receive a fastener, such as a bone screw (not shown). The bone plate of the bone fixation apparatus also includes plurality of interconnecting portions 218 that join two boundary portions 214 to one another. As shown in FIGS. 5 and 6, the bone fixation apparatus 200 may be applied to a bone 220 to reduce and stabilize one more fractures 222 in the bone.

In the embodiment of FIGS. 5 and 6, the arrangement of boundary portions 214 and interconnecting portions 218 in the bone plate 212 may resemble the arrangement of the bone plate 12 of FIGS. 1 and 2 or the arrangement of the bone plate 112 of FIGS. 3 and 4 or any other arrangement of boundary portions and interconnecting portions. Unlike the arrangement of boundary portions 14 and interconnecting portions 18 in the bone plate 12 or the arrangement of boundary portions 114 and interconnecting portions 118 in the bone plate 112, however, each of the boundary portions 214 is not free of any structure extending radially from the boundary portion other than one or more of the interconnecting portions 218. Specifically, certain of the boundary portions 214 have tabs 240 that extend away from the boundary portions and that may wrap around a bone 220 to an extent that the tabs overlie or are overlain by other portions of the bone plate 212, as shown, for example, in FIG. 6.

More particularly, the bone plate 212 includes seven boundary portions 214. Only five boundary portions 214a-214e of the seven boundary portions 214 are visible in FIG. 5. The bone plate 212 also includes eight interconnecting portions 218. Only four interconnecting portions 218a-218d are visible in FIG. 5. Boundary portions 214c and 214d are wider than boundary portions 214a and 214b. Similarly, interconnecting portions 218c and 218d are wider than interconnecting portions 218a and 218b. Tab 240a extends radially from boundary portion 214b, and tab 240b extends radially from boundary portion 214d. Various boundary portions 214 and interconnecting portions 218 are joined together in an array with a generally zigzag configuration. For example, boundary portions 214b, 214c, and 214d and interconnecting portions 218b and 218c are joined together in an array with a generally zigzag configuration.

As can be seen in FIG. 6, boundary portion 214c lies in a plane 224. Boundary portion 214f (visible only in FIG. 6) is disposed in a plane 226 adjacent the left side of the bone plate 212 and the bone 220, as viewed in FIG. 6, and oriented transverse to the plane 224 in which the boundary portion 214c is disposed. Boundary portion 214d disposed in a plane 228 adjacent the right side of the bone plate 212 and the bone 220, as viewed in FIG. 6. The boundary portion 214d is also spaced laterally from boundary portion 214f across the width of the bone plate 212 and the thickness or width of the bone 220 but is aligned with the boundary portion 214f longitudinally along the length of the bone plate 212 and the length of the bone 220. The plane 228 in which the boundary portion 214d is disposed is spaced laterally apart from the plane 226 in which the boundary portion 214f is disposed by the width of the bone plate 212 and thickness or width of the bone 220, but the planes 226 and 228 are substantially parallel to one another. The plane 228 in which the boundary portion 214d is disposed is transverse, however, to the plane 224 in which the boundary portion 214c is disposed. on a side of the bone 220 opposite the boundary portion 214d.

Tab 240c extends radially from boundary portion 214f and is disposed in the plane 226. Tab 240c simply extends radially from the boundary portion 214f and does not extend to any side of the bone 220 other than the side adjacent the boundary portion 214f. Tab 240b, on the other hand, wraps around the bone 220 so that the tab 240b is disposed partially in the plane 228 and partially in a plane 230 that is transverse to the plane 228. The tab 240b is thus disposed on a side of the bone opposite the boundary portion 214c. The plane 230 in which the tab 240b is partially disposed is substantially parallel to the plane 224 in which the boundary portion 214c is disposed. Boundary portion 214c thus overlies or is overlain by tab 240b.

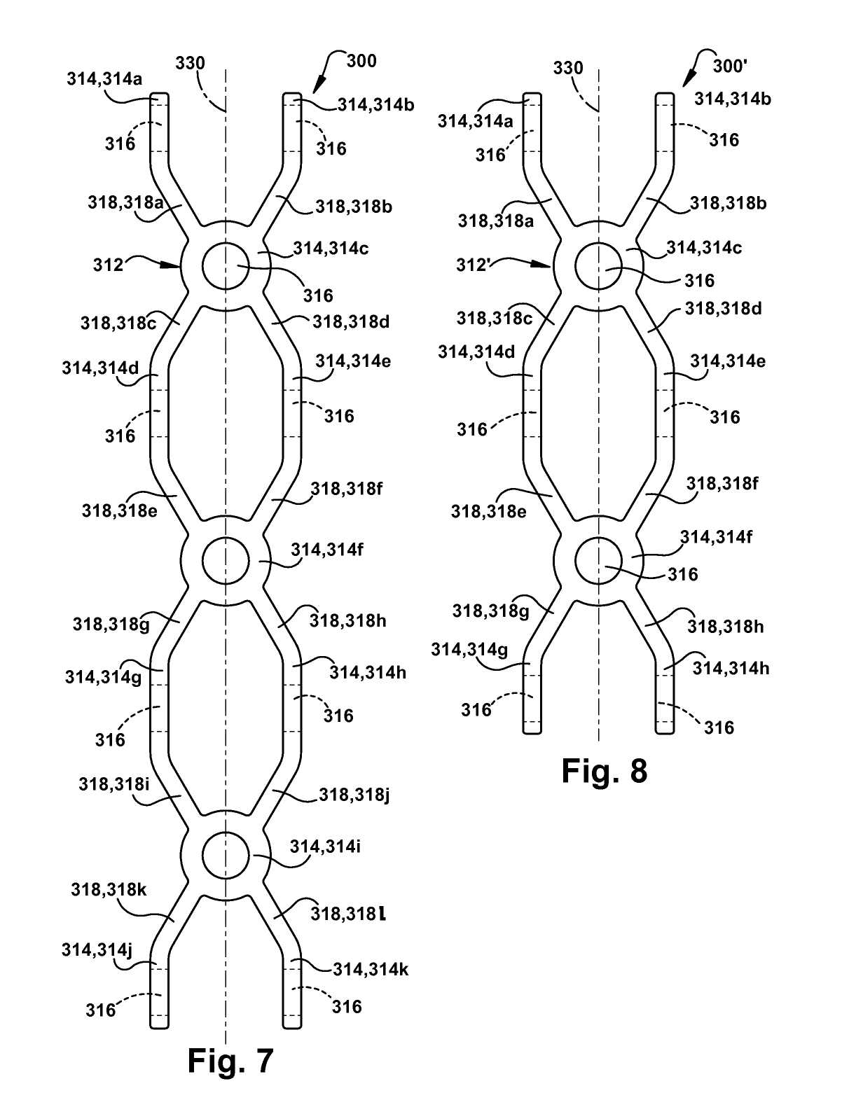

Although the bone fixation apparatus 10 is shown in FIGS. 1-2 as having six boundary portions 14 and five interconnecting portions 18 and although the bone fixation apparatus 100 is shown in FIGS. 3-4 as having eight boundary portions 114 and eight interconnecting portions 118, other numbers of boundary portions 14 and 114 and other numbers of interconnecting portions 18 and 118, either more or fewer, may be used. By way of example, FIG. 7 illustrates a bone fixation apparatus 300 in accordance with a fourth example of the present invention. FIG. 8 illustrates a bone fixation apparatus 300′ that is a modified version of the bone fixation apparatus 300. Both the bone fixation apparatus 300 and the bone fixation apparatus 300′ comprise a bone plate 312, 312′ that includes a plurality of boundary portions 314. Each boundary portion 314 defines and surrounds an opening 316 that is configured and dimensioned to receive a fastener, such as a bone screw (not shown). The bone plate 312, 312′ of the bone fixation apparatus 300, 300′ also includes plurality of interconnecting portions 318 that join two boundary portions 314 to one another. Both the bone fixation apparatus 300 and the bone fixation apparatus 300′ may be applied to a bone (not shown) to reduce and stabilize one or more fractures in the bone.

Both the bone fixation apparatus 300 and the bone fixation apparatus 300′ resemble the bone fixation apparatus 100 of FIGS. 3 and 4. In the embodiment of FIG. 7, the plurality of boundary portions 314 in the bone plate 312 comprises eleven boundary portions 314a, 314b, 314c, 314d, 314e, 314f, 314g, 314h, 314i, 314j, and 314k. The plurality of interconnecting portions 318 in the bone plate 312 comprises twelve interconnecting portions 318a, 318b, 318c, 318d, 318e, 318f, 318g, 318h, 318i, 318j, 318k, and 318l. Each of interconnecting portions 318a-318l has two opposed ends and a length extending between the opposed ends. Each of the interconnecting portions 318a-318l is joined at one of its opposed ends to a first boundary portion 314 and at the other of its opposed ends to second boundary portion. For example, the interconnecting portion 318a is joined at one end to the boundary portion 314a and at its other end to the boundary portion 314c. The eleven boundary portions 314 and the twelve interconnecting portions 318 are all formed in one piece from a homogeneous material. The material is a relatively rigid bio-compatible material, such as medical grade stainless steel or titanium.

The boundary portions 314 and interconnecting portions 318 are joined together in an array that provides the bone plate 312 with a longitudinal series of configurations shaped like X's or, viewed differently, with a pair of zigzag configurations. In particular, the boundary portions 314a and 314b are spaced apart by a distance that will accommodate the thickness or width of a bone (not shown) disposed between the boundary portions. The boundary portions 314a and 314b are also presented generally toward one another. The boundary portion 314c is positioned laterally between the boundary portions 314a and 314b and is spaced longitudinally from the boundary portions 314a and 314b along the length of the bone plate 312. The boundary portion 314c also extends laterally in a plane transverse to the planes in which the boundary portions 314a and 314b extend.

As previously described, the interconnecting portion 318a extends between and joins together the boundary portion 314a and the boundary portion 314c. The interconnecting portion 318b extends between and joins together the boundary portion 314b and the boundary portion 314c. Because the boundary portion 314c lies in a plane that extends transverse to the planes in which the boundary portions 314a and 314b lie, the interconnecting portions 318a and 318b can curve or bend around a bone (not shown). In addition, because the boundary portion 314c is spaced apart from the boundary portions 314a and 314b along the length of the bone plate 312, the interconnecting portions 318a and 318b extend across the bone plate at angles to a longitudinal central axis 330 of the bone plate. As viewed in FIG. 7, the interconnecting portion 318a zigs to the right, and the interconnecting portion 318b zags to the left.

The boundary portion 314d is spaced from the boundary portion 314c along the length of the bone plate 312 and is disposed in the same plane, adjacent the left side of the bone plate, as the boundary portion 314a. The interconnecting portion 318c extends between and joins together the boundary portion 314c and the boundary portion 314d. Because the boundary portion 314d lies in a plane that extends transverse to the plane in which the boundary portion 314c lies, the interconnecting portion 318c can curve or bend around a bone. In addition, because the boundary portion 314d is spaced apart from the boundary portion 314c along the length of the bone plate 312, the interconnecting portion 318c extends across the bone plate at an angle to the longitudinal central axis 330 of the bone plate. As viewed in FIG. 7, the interconnecting portion 318c zags to the left.

The boundary portion 314e is spaced longitudinally from the boundary portion 314c along the length of the bone plate 312 and is disposed in the same plane, adjacent the right side of the bone plate, as the boundary portion 314b. The boundary portion 314e is spaced laterally from the boundary portion 314d across the width of the bone plate 312 but is aligned with the boundary portion 314d longitudinally along the length of the bone plate 312, as can be seen in FIG. 7. The plane in which the boundary portion 314e is disposed is spaced laterally apart from the plane in which the boundary portion 314d is disposed by the width of the bone plate 312, but the planes are substantially parallel to one another. The plane in which the boundary portion 314e is disposed is transverse, however, to the plane in which the boundary portion 314c is disposed.

The interconnecting portion 318d extends between and joins together the boundary portion 314c and the boundary portion 314e. Because the boundary portion 314e lies in a plane that is transverse to the plane in which the boundary portion 314c lies, the interconnecting portion 318d can curve or bend around a bone. In addition, because the boundary portion 314e is spaced apart from the boundary portion 314c along the length of the bone plate 312, the interconnecting portion 318d extends across the bone plate at an angle to the longitudinal central axis 330 of the bone plate. As viewed in FIG. 7, the interconnecting portion 318d zigs to the right.

As viewed in FIG. 7, boundary portion 314c is located in a central position relative to the boundary portions 314a, 314b, 314d, and 314e along the length of the bone plate 312. The interconnecting portions 318a, 318b, 318c, and 318d extend radially from the boundary portion 314c. The boundary portions 314a, 314b, 314c, 314d, and 314e and the interconnecting portions 318a, 318b, 318c, and 318d are thus arranged in a configuration shaped like an “X”. In addition, the boundary portions 314a, 314c, and 314d and the interconnecting portions 318a and 318c are arranged in a zigzag configuration. Further, the boundary portions 314b, 314c, and 314e and the interconnecting portions 318b and 318d are arranged in a zigzag configuration that is opposed to the zigzag configuration of the boundary portions 314a, 314c, and 314d and the interconnecting portions 318a and 318c. Each of the boundary portions 314a-314e is joined only to one or more of interconnecting portions 318a-318f. Each of the boundary portions 314a-314e is free of any structure extending radially from the boundary portion other than one or more of interconnecting portions 318a-318f.

The remaining boundary portions 314f through 314k and interconnecting portions 318e through 318l are joined together in an array that is an extension of the array in which the boundary portions 314a-314e and the interconnecting portions 318a-318d are joined together. As previously described, the overall pattern is an array that provides the bone plate 312 with a longitudinal series of configurations shaped like X's or, viewed differently, with a pair of zigzag configurations.

The bone fixation apparatus 300′ is identical to the bone fixation apparatus 300 in all respects except that the bone plate 312′ of the bone fixation apparatus 300′ lacks boundary portions 314i through 314k and interconnecting portions 318i through 318l. The bone fixation apparatus 300′ and the bone plate 312′ are thus different in length than, i.e., shorter than, the bone fixation apparatus 300 and the bone plate 312. This difference in lengths may be either predetermined or determined at the time the bone fixation apparatus 300′ is placed in use. Specifically, a surgeon implanting the bone plate 312 in a patient may cut out or trim away boundary portions 314i through 314k and interconnecting portions 318i through 318l from the bone plate 312 to produce the bone plate 312′ and the bone fixation apparatus 300′. The surgeon may elect to cut or trim any number of boundary portions 314 and/or interconnecting portions 318 as required to have the bone plate 312 fit a particular bone or bone fracture. The number of boundary portions 314 and interconnecting portions 318 cut out or trimmed away may be greater or smaller than the number shown in FIG. 8 and described above.

From the above description of the invention, those skilled in the art will perceive improvements, changes and modifications. Such improvements, changes, and/or modifications within the skill of the art are intended to be covered by the appended claims.

1. A bone fixation apparatus comprising:
(a) a plurality of boundary portions, each boundary portion defining and surrounding an opening for a fastener; and
(b) a plurality of interconnecting portions, each interconnecting portion having two opposed ends and a length extending between the opposed ends, each interconnecting portion being joined at one of its opposed ends to a first boundary portion and at the other of its opposed ends to a second boundary portion so as to extend radially from the first and second boundary portions, each boundary portion being joined only to at least one interconnecting portion and being free of any structure extending radially from said each boundary portion other than said at least one interconnecting portion,
three of the plurality of boundary portions and two of the interconnecting portions being arranged in a zigzag configuration with one of the three boundary portions being located in a central position and the two interconnecting portions extending radially from the boundary portion in the central position to two different ones of the three boundary portions, the boundary portion in the central position being disposed in a first plane and the two different ones of the three boundary portions being disposed in a second plane.

2. A bone fixation apparatus according to claim 1 wherein the second plane is substantially parallel to and spaced from the first plane.

3. A bone fixation apparatus according to claim 1 wherein the second plane intersects the first plane and is oriented substantially perpendicular to the first plane.

4. A bone fixation apparatus comprising:
(a) a plurality of boundary portions, each boundary portion defining and surrounding an opening for a fastener; and
(b) a plurality of interconnecting portions, each interconnecting portion having two opposed ends and a length extending between the opposed ends, each interconnecting portion being joined at one of its opposed ends to a first boundary portion and at the other of its opposed ends to a second boundary portion so as to extend radially from the first and second boundary portions, each boundary portion being joined only to at least one interconnecting portion and being free of any structure extending radially from said each boundary portion other than said at least one interconnecting portion,
five of the plurality of boundary portions and four of the interconnecting portions being arranged in an X configuration with one of the five boundary portions being located in a central position and the four interconnecting portions extending radially from the boundary portion in the central position to four different ones of the five boundary portions, the boundary portion in the central position being disposed in a first plane and the four different ones of the five boundary portions being disposed in second planes transverse to the first plane.

5. A bone fixation apparatus comprising:
(a) a plurality of boundary portions, each boundary portion defining and surrounding an opening for a fastener;
(b) a plurality of interconnecting portions, each interconnecting portion having two opposed ends and a length extending between the opposed ends, each interconnecting portion being joined at one of its opposed ends to a first boundary portion and at the other of its opposed ends to a second boundary portion so as to extend radially from the first and second boundary portions; and
(c) a plurality of tab portions, each tab portion extending radially from a single boundary portion,
each boundary portion being joined only to at least one of the tab portions and the interconnecting portions, each boundary portion also being free of any structure extending radially from said each boundary portion other than said at least one of the tab portions and the interconnecting portions,
three of the plurality of boundary portions and two of the interconnecting portions being arranged in a zigzag configuration with one of the three boundary portions being located in a central position and the two interconnecting portions extending radially from the boundary portion in the central position to two different ones of the three boundary portions, the boundary portion in the central position being disposed in a first plane and the two different ones of the three boundary portions being disposed in a second plane, one of the tab portions extending radially from one of the two different ones of the three boundary portions and being at least partially disposed in a third plane spaced from and generally parallel to the first plane.

6. A bone fixation apparatus according to claim 1 wherein said two of the interconnecting portions are free of any opening.

7. A bone fixation apparatus according to claim 1 wherein said three of the plurality of boundary portions comprise first, second and third boundary portions and wherein said two of the interconnecting portions comprise first and second interconnecting portions, said one of the three boundary portions located in a central position being the second boundary portion, the first interconnecting portion extending radially from the second boundary portion to the first boundary portion, the second interconnecting portion extending radially from the second boundary portion to the third boundary portion, said bone fixation apparatus further comprising a fourth boundary portion, a fifth boundary portion, a third interconnecting portion, and a fourth interconnecting portion, the third interconnecting portion extending radially from the third boundary portion to the fourth boundary portion, the fourth interconnecting portion extending radially from the fourth boundary portion to the fifth boundary portion, the first, second, third, fourth, and fifth boundary portions and the first, second, third, and fourth interconnecting portions thereby being arranged in two zigzag configurations with the third boundary portion being included in both zigzag configurations.

8. A bone fixation apparatus according to claim 4 wherein said four of the interconnecting portions are free of any opening.

9. A bone fixation apparatus according to claim 4 wherein said five of the plurality of boundary portions comprise first, second, third, fourth, and fifth boundary portions and wherein said four of the interconnecting portions comprise first, second, third, and fourth boundary portions, said one of the five boundary portions located in a central position being the third boundary portion, the first interconnecting portion extending radially from the third boundary portion to the first boundary portion, the second interconnecting portion extending radially from the third boundary portion to the second boundary portion, the third interconnecting portion extending radially from the third boundary portion to the fourth boundary portion, the fourth interconnecting portion extending radially from the third boundary portion to the fifth boundary portion, said bone fixation apparatus further comprising a sixth boundary portion, a seventh boundary portion, an eighth boundary portion, a fifth interconnecting portion, a sixth interconnecting portion, a seventh interconnecting portion, and an eighth interconnecting portion, the fifth interconnecting portion extending radially from the fourth boundary portion to the sixth boundary portion, the sixth interconnecting portion extending radially from the fifth boundary portion to the sixth boundary portion, the seventh interconnecting portion extending radially from the sixth boundary portion to the seventh boundary portion, the eighth interconnecting portion extending radially from the sixth boundary portion to the eighth boundary portion, the first, second, third, fourth, fifth, sixth, seventh, and eighth boundary portions and the first, second, third, fourth, fifth, sixth, seventh, and eighth interconnecting portions thereby being arranged in two X configurations with the fourth and fifth boundary portions being included in both X configurations.
